(19) United States
(12) Patent Application Publication
(10) Pub. No.: US 2020/0268115 A1
(43) Pub. Date: Aug. 27, 2020

(54) MICRODISPENSING JEWELRY

(71) Applicant: Xinova, LLC, Seattle, WA (US)

(72) Inventor: Michael Keoni MANION, Seattle, WA (US)

(73) Assignee: Xinova, LLC, Seattle, WA (US)

(21) Appl. No.: 16/642,984

(22) PCT Filed: Oct. 17, 2018
(86) PCT No.: PCT/US18/56180

§ 371 Date: Feb. 28, 2020

Related U.S. Application Data
(60) Provisional application No. 62/577,126, filed on Oct. 25, 2017.

(51) Int. Cl. A44C 15/00; A61B 5/0205

(52) U.S. Cl. CPC A44C 15/002; A61B 5/021; A61B 5/02055

(57) ABSTRACT

Systems, devices, and methods for dispensing a fragrant liquid on a person are described. A system includes a computing device and a personal ornament to be worn by the person. The computing device includes information related to a delivery schedule for dispensing a fragrant liquid. The personal ornament includes a container configured to store a fragrant liquid, a microdispenser fluidly connected to the container, wherein the microdispenser is configured to deliver bursts of the fragrant liquid over a period of time, and a controller operably connected to the microdispenser.

### BACKGROUND

Perfume has been widely used for thousands of years to provide a pleasant smell or to mask an odor. It is typically applied at pulse points, such as behind the ears, at the nape of the neck, and on the insides of the wrists. The pulse points act to warm the perfume and provides for a prolonged release of fragrance.

However, the longevity of the fragrance is limited, and its effectiveness diminishes with time. The longevity of a perfume, as well as the cost of the perfume, are typically correlated to the percentage of essential oils in the perfume. The perfume extracts, such as Eau de Parfum, that have a higher concentration of essential oils are typically more expensive than the less concentrated extracts, such as Cologne or Eau de Toilette. The inexpensive alternatives typically do not last as long as the expensive extracts due to their lower concentration of the essential oils.

### SUMMARY

A personal ornament for dispensing a fragrant liquid is disclosed. In certain implementations, the personal ornament may include: a container configured to store a fragrant liquid; a microdispenser fluidly connected to the container, wherein the microdispenser is configured to deliver bursts of the fragrant liquid over a period of time; and a controller operably connected to the microdispenser and configured to provide instructions to the microdispenser to deliver the fragrant liquid according to a delivery schedule.

A method of dispensing a fragrant liquid on a person is also described. In certain implementations, the method includes: determining, by a controller integrated into a personal ornament worn by the person, timing for dispensing a fragrant liquid based upon a delivery schedule; sending, from the controller to a microdispenser, instructions for the microdispenser to deliver at least one burst of the fragrant liquid; receiving, at the microdispenser, the fragrant liquid from a container fluidly attached to the microdispenser; and dispensing, by the microdispenser, an amount of the fragrant liquid onto a portion of skin of the person.

A system for dispensing a fragrant liquid on a person is also described. In certain implementations, the system includes a computing device that includes information related to a delivery schedule for dispensing a fragrant liquid on a person and a personal ornament worn by the person. In some examples, the personal ornament includes a container configured to store a fragrant liquid, a microdispenser fluidly connected to the container, wherein the microdispenser is configured to deliver bursts of the fragrant liquid over a period of time, and a controller operably connected to the microdispenser. The controller may be configured to establish communications with the computing device, receive the delivery schedule from the computing device, and provide instructions to the microdispenser to deliver the fragrant liquid according to the delivery schedule.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the present disclosure. In the drawings:

FIG. 1 depicts an illustrative personal ornament for dispensing a fragrant liquid in accordance with an embodiment.

FIG. 2 depicts a detailed component level diagram for a personal ornament in accordance with an embodiment.

FIG. 3 depicts a system of multiple devices in accordance with an embodiment.

FIG. 4 depicts a set of personal ornaments in accordance with an embodiment.

FIG. 5 depicts a flow diagram of an illustrative process for distributing a fragrant liquid using one or more personal ornaments in accordance with an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

The present disclosure is directed to personal ornaments, such as jewelry, that are configured to periodically dispense measured amounts or controlled dosages of a fragrant liquid, such as a perfume, by using a controlled burst of the fragrant liquid. In some examples, the measured amounts of fragrant liquid may be picoliter amounts, nanoliter amounts, or microliter amounts. The jewelry may include smart components, such as a controller in communication with a microdispenser configured to dispense very small amounts (e.g., microliter amounts) of the perfume over time. Through such control, the jewelry (or the person wearing the jewelry) may precisely control the amount and intensity of the perfume over much longer periods of time as compared to traditional perfume application.

The jewelry as described herein may have one or more of various form factors. For example, the jewelry may include one or more of earrings, necklaces, and bracelets. In certain implementations, the jewelry may also include one or more of a ring, a brooch, a hair clip, an armlet, a hairpin, a fascinator, a chatelaine, a cufflink, an anklet, a body chain, a pendant, a piercing, and a bangle. It should be noted that the terms personal ornament and jewelry as used herein may be interpreted as having a similar meaning and may include any specific form factor as listed above.

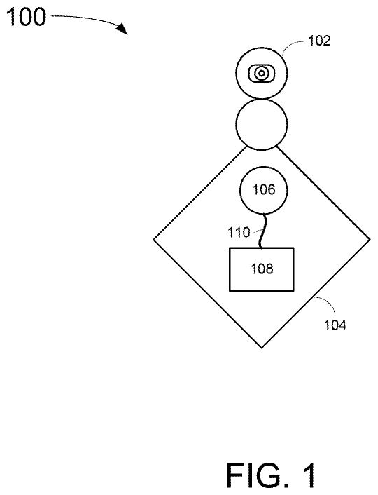

The jewelry itself may have a fairly straightforward and classical design such that the jewelry may be worn as normal jewelry without calling unwanted attention to the wearer. FIG. 1 illustrates a sample piece of jewelry incorporating the features as described herein. For example, an earring 100 may include various components for dispensing perfume onto a portion of the body of a person wearing the earring, e.g., the wearer's neck. In some examples, the earring 100 may be made with materials similar to those used in standard jewelry design and manufacture. For examples, the earring 100 may be made from a precious metal, such as gold, silver, or platinum, or from a more common material, such as stainless steel or various plastics. The earring 100 may include a standard fastener such as a post and back fastener 102 configured to hold the earring on the wearer's ear using a friction fit. In certain implementations, the earring 100 may also include an additional decoration 104 such as a gemstone, a hoop, a bangle, and/or other common visual decorations.

The earring 100 may also include various components for facilitating application of perfume to the person wearing the earring. In certain implementations, the earring 100 may include an internal container or reservoir 106 configured to hold a quantity of the perfume. In some examples, the reservoir 106 may be sized to hold an amount of perfume to provide for regular application throughout a specific time period. For example, the reservoir 106 may be configured to hold enough perfume for application every twenty minutes over a period of twelve hours. To accommodate such an amount, the reservoir 106 may be sized appropriately. In certain implementations, the reservoir 106 may be slightly oversized to accommodate an extra amount of the perfume in the event that the wearer chooses to include extra application of the perfume contained therein. For example, the size of the reservoir 106 may range from about 1 mL to 2 mL, from about 2 mL to 3 mL, and about 3 mL or greater.

It should be noted that, as used herein, perfume refers to various types of perfumes including various concentrations of essential oils. For example, as used herein, perfume may include after-shave, Cologne, Eau de Toilette, Eau de Parfum, and perfume extract.

The reservoir 106 may be made from a material that may withstand normal activity and is not easily punctured. For example, the reservoir 106 may be made from a thermoplastic resin using a thermo-forming process to produce a reservoir 106 that is resistant to compressing forces and twisting forces likely to be exerted on the earring 100 when worn. The reservoir 106 may also be made such that the perfume contained therein is protected from dirt, water, and other debris that the earring 100 might be exposed to in normal daily use.

The earring 100 may also include a microdispenser 108. In certain implementations, the microdispenser 108 may be integrated into a microchip that is located on or within the underside of the earring 100 such that the microdispenser is positioned adjacent to the wearer's skin. The microdispenser 108 may be configured to deliver a series of microdrops of medication to the wearer's skin at a certain frequency for a particular period of time. For example, the microdispenser 108 may be configured to deliver a series of 50 picoliter microdrops at a frequency of about 20 Hz, thereby delivering about 5 microliters of perfume per application. The microdispenser 108 may be configured to deliver the bursts of perfume with enough force to deliver the medication to the surface of the wearer's skin.

In certain implementations, the microdispenser 108 may be configured to deliver microdrops sized from about 25 picoliters to about 75 picoliters. In some examples, the microdispenser 108 may be configured to deliver about 2.5 to 7.5 microliters of perfume per application. In certain implementations, the microdispenser 108 may be configured to operate at a frequency of about 10 Hz to about 50 Hz.

In certain implementations, the microdispenser 108 may be a piezoelectric microdispenser configured to produce a burst or otherwise dispense an amount of fragrant liquid such as perfume in response to an electrical impulse causing a piezoelectric element to eject a portion of the liquid from the microdispenser. In other implementations, the microdispenser 108 may be a thermoelectrically activated microdispenser configured to produce a burst or otherwise dispense an amount of the fragrant liquid in response to a thermally reactive element increasing in temperature, causing a gas bubble to expand and eject a portion of the liquid from the microdispenser.

The reservoir 106 and the microdispenser 108 may be fluidly connected by a fluid channel 110. The fluid channel 110 may be integrated into the earring 100 itself. The fluid channel 110 may be attached to the reservoir 106 and the microdispenser 108 to form a watertight seal, thereby preventing dirt, water, or other debris from contaminating the perfume in the reservoir or potentially clogging the microdispenser.

In some implementations, the location and spray direction of the microdispenser 108 may be determined and arranged such that the perfume is delivered to a specific point on the wearer's body. In order to maintain a traditional appearance for the earring 100, the microdispenser may be placed in a discrete location that is not readily visible or apparent when looking at the earring when worn. For example, with earring 100, the microdispenser 108 may be positioned to spray the perfume from the back of the earring onto the wearer's neck or behind their ear.

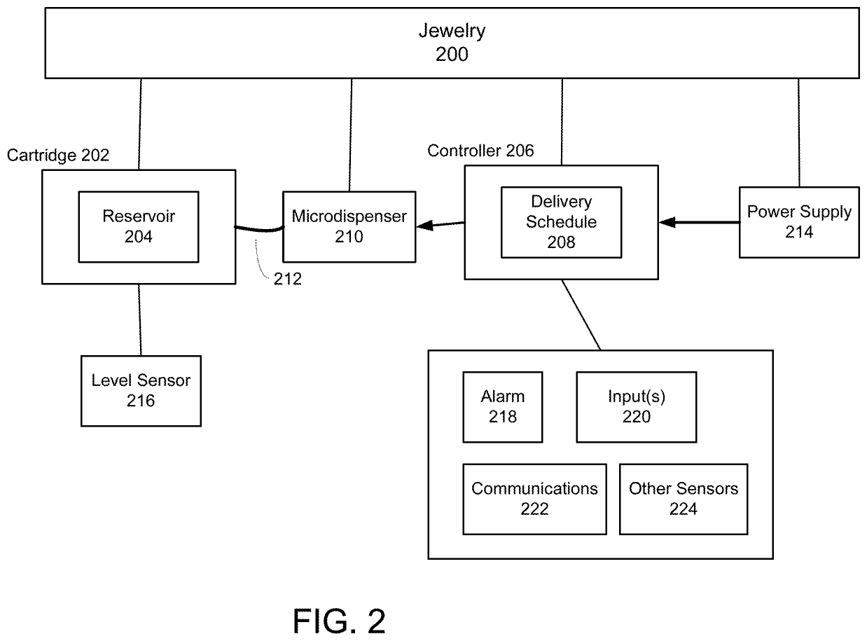

FIG. 1 illustrates the basic components included in the dispensing jewelry as taught and described herein. FIG. 2 illustrates a more detailed component level view of jewelry for dispensing perfume including various optional features and functionality described herein.

As shown in FIG. 2, a piece of jewelry 200 may include a replaceable cartridge 202. The cartridge 202 may include a reservoir 204 configured to hold a quantity of perfume. In certain implementations, the jewelry 200 may be configured to quickly receive a new cartridge 202, thereby providing a full reservoir 204 including a new perfume. However, it should be noted that including a replaceable cartridge 202 is provided by way of example only. In certain implementations, the reservoir 204 may be refillable and include, for example, a fill port or other similar opening or aperture into which additional perfume may be inserted.

The jewelry 200 may also include a controller 206. The controller 206 may include, for example, a microprocessor or other similar processing device. The controller 206 may also include a computer-readable storage medium that is configured to store a delivery schedule 208. In certain implementations, the delivery schedule 208 may include instructions incorporating quantity and timing information for application of the perfume stored, for example, in reservoir 204. Various types of delivery schedules 208 may be used by the jewelry 200 depending upon various factors such as the wearer's personal preferences, what type of perfume is being used, the time of day, the activity level of the wearer, and other similar factors. For example, the delivery schedule 208 may include a continuous release of the perfume at a steady rate throughout the wearer's working hours (e.g., release every half hour between the hours of 9 AM and 5 PM). After 5 PM, the delivery schedule 208 may indicate that the release is to be increased to every twenty minutes if, for example, the wearer is going out on a date or to another similar social function and wants to increase their perfume application. In another example, the delivery schedule 208 may include a complex release schedule, such as distributing more perfume when the jewelry 200 is initially put on and tapering the amount of perfume delivered to a lower amount throughout the day. In some examples, the delivery schedule 208 may include one or more periods of time (e.g., more than one hour) during which no perfume is delivered to the wearer.

The jewelry 200 may further include at least one microdispenser 210. The microdispenser 210 may be operably connected to the controller 206 and configured to receive dispensing instructions according to the delivery schedule 208. The microdispenser 210 may also be connected to the cartridge 202 via a fluid channel 212.

In certain implementations, the jewelry 200 may also include a power supply 214 configured to provide power to various components in the collar such as the controller 206 and/or the microdispenser 210. In certain implementations, the controller 206 may be configured to provide modulated power to the microdispenser 210 along with dispensing control signals. In some examples, the power supply 214 may be a rechargeable battery. For example, the power supply 214 may include a battery configured to be wirelessly charged using a standard wireless charging protocol (e.g., Qi wireless charging). In some implementations, the power supply 214 may include kinetic recharging. In such an example, the movement of the person wearing the jewelry 200 may be used to create energy that may be stored in a battery or other similar energy storage medium contained within, or operably connected to, the power supply 214.

As shown in FIG. 2, in certain implementations, various components within the jewelry 200 may include one or more additional features. For example, the cartridge 202 may include various additional features, such as a level sensor 216. The level sensor 216 may be configured to provide an indication of the status of the cartridge 202 and an indication of whether the cartridge should be replaced or refilled. Depending upon the implementation, the level sensor 216 may be configured to provide a status signal to the controller 206. Alternatively and/or additionally, the level sensor 216 may include an indicator, such as a visual indicator or audible alarm, used to indicate that the perfume level is below a certain threshold.

The controller 206 may also include various additional components and features. For example, the controller 206 may include, or be operably connected to, an alarm 218. The alarm 218 may be configured to provide an audible and/or visual indication if one or more criteria are met. For example, if the cartridge 202 has become disconnected from the jewelry 200, the alarm 220 may output an audible and/or visual notification that something is wrong with the jewelry. As described in further detail below, the alarm 218 may also be configured to operate with other features of the controller 206 and/or jewelry 200 to provide notification of the current status of the jewelry 200. For example, the alarm 218 may be configured to output an alarm if the controller receives an indication from the level sensor 216 that the perfume level has fallen below a certain threshold.

The controller 206 may further include one or more inputs 220. In certain implementations, the inputs 220 may include one or more activation/deactivation buttons configured to function as power buttons for the jewelry 200. In certain implementations, the inputs 220 may further include a manual activation or dispenser control input. Such an input may provide for a person to control manual dispensing of the perfume in addition to the delivery schedule 208. In certain implementations, upon activation of a manual dispensing control, the control may wait a period of time (e.g., 15 seconds) to allow the wearer to remove their hand from the jewelry 200 and to provide for a discrete application of the dispensed perfume. In some examples, the manual dispensing control may be configured to deliver a set amount of the perfume or to provide a variable amount of perfume that is related to how long the wearer pressed the manual dispensing control.

The inputs 220 may also include a volume or dispensing amount control. For example, the inputs 220 may include a dial or other similar adjustable control that provides the wearer with the ability to change the amount of perfume that is dispensed by the jewelry 200.

The controller 206 may further include an auto-shutoff feature 224. The auto-shutoff feature 224 may be configured to shut off the controller 208 and the microdispenser 210 if, for example, the cartridge 202 is empty or has been removed, or if the collar has been removed from the wearer.

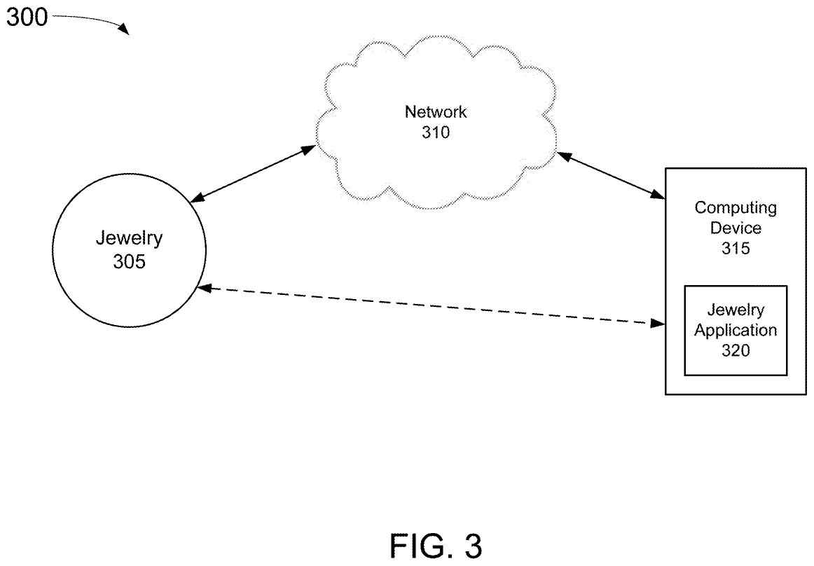

In certain implementations, the controller 206 may also include a communication interface 222. The communication interface 222 may include a wireless transceiver configured to establish a wireless communication connection with an external network and/or a remote computing device. For example, the communication interface 222 may be configured to establish a Bluetooth connection, a WI-FI connection, a mesh network connection, a near-field communication connection, or another similar wireless connection. Communications between jewelry 200 and an external network and/or a remote computing device is described in additional detail in the description of FIG. 3 below.

In some examples, the controller 206 may also include other sensors 224. For example, the other sensors 224 may include a temperature sensor configured to monitor the temperature of the person wearing the jewelry 200, an activity sensor configured to monitor physical activity of the wearer, and other similar sensors. Based upon the output of the additional sensors 224, the controller 206 may be configured to alter the delivery schedule 208 or to otherwise alter the dispensing of the perfume. For example, if the temperature sensor indicates an increase in the wearer's body temperature, and the activity sensor indicates an increase in the wearer's activity, the controller may be programmed to interpret these changes as an indication that the wearer is exercising and may increase the perfume dosage to counteract any potential unpleasant odors that may result from sweating due to the exercise.

It should be noted that the arrangement of the components as shown in FIG. 2 is by way of example only. This is particularly true for the various additional components. For example, the level sensor 216 may be integrated with or operably connected to the controller 206. Information related to the level sensor 216 (e.g., remaining perfume level) may be output or sent to another computing device via the communication interface 222.

It should also be noted that the various additional components as described above may be optional depending upon the implementation of the jewelry 200. Depending upon the design, intended use, and available power for the jewelry 200, the amount and type of additional features included in the collar may vary accordingly.

FIG. 3 illustrates a sample system 300 for communication between at least one piece of jewelry and a remote computing device. As described above, a piece of jewelry 305 may include a communication interface configured to operably connect to a remote network, such as network 310. In certain implementations, the network 310 may be implemented as a wireless network. For example, the network 310 may be a wide area network, such as a WI-FI network. In other examples, the network may be a local area network such as a mesh network. A remote computing device 315 may also be configured to connect to the network 310, thereby establishing a connection with the jewelry 305. In certain implementations, the remote computing device 315 may be a smartphone used by the person wearing the jewelry 305. In some examples, the remote computing device 315 may include an application 320 configured to interact with various features on the jewelry 305. For example, the application 320 may receive information from the jewelry 305 regarding various information related to the operation of the jewelry 305 such as perfume levels, dispensing history, power level, previous alarm information, and other related information. The wearer may also use the application 320 to provide an updated delivery schedule, initiate a manual dispensing, clear alarm information, check perfume levels, and perform additional tasks related to the operation of the jewelry 305.

Depending upon the communication capabilities of the jewelry 305, a direct connection may be established between the jewelry and the computing device 315. For example, the jewelry 305 and computing device 315 may be configured to connect using Bluetooth, near field communications, or another similar short-range communication protocol.

It should be noted that a single piece of jewelry 305 is shown in FIG. 3 by way of example. In actual practice, a person may wear multiple pieces of j ewelry that are each configured to dispense perfume using the techniques and processes as described herein. In such an arrangement, each individual price of jewelry may also be connected to network 310 or directly to computing device 315. Additionally, the pieces of jewelry may be configured to establish or connect to a small area network, such as a mesh network, thereby allowing the individual pieces of jewelry to interact directly. This may be used to synchronize delivery of the perfume or to layer multiple scents at the same time. For example, a person may wear a pair of earrings. Each earring may include a reservoir filled with a different perfume. In some implementations, the different perfumes may be complimentary scents and may work together to provide a layering effect. The individual controllers for each of the earrings may be able to communicate directly with each other, synchronizing the dispensing of each perfume to provide for a complex and layered scent profile for the wearer.

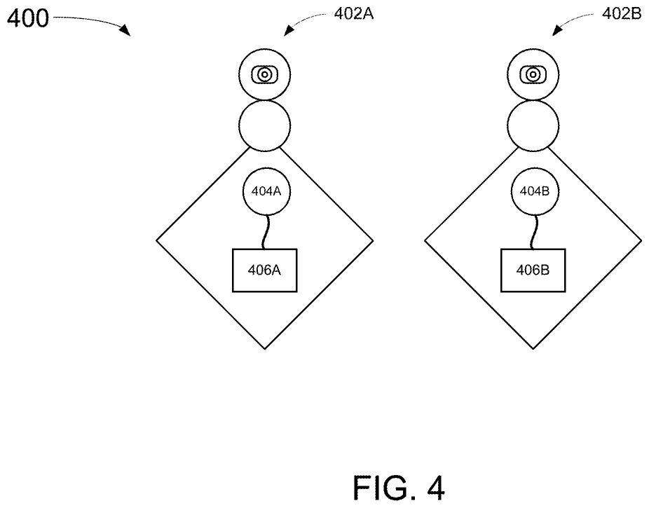

For example, as illustrated in FIG. 4, a pair of earrings 400 may include multiple individual earrings 402A and 402B. A first earring 402A may include a first reservoir 404A, and a second earring 402B may include a second reservoir 404B. In certain implementations, each reservoir 404A, 404B may contain the same type of perfume. However, as noted above, in some examples, each reservoir 404A, 404B may contained a different perfume. Each reservoir may be connected to a separate microdispenser 406A, 406B.

Additionally, rather than a single microdispenser as has been described herein, a piece of jewelry may be designed such that it includes an array of microdispensers. Such a design may increase the amount of perfume that is applied to a person wearing the jewelry while reducing the amount of time required to apply the perfume. For example, an array of 3×3 microdispensers may be incorporated into a piece of jewelry.

It should also be noted that a single reservoir and microdispenser pair is shown herein by way of example only. Depending upon the size and design of a piece of jewelry, the jewelry may include multiple reservoirs and/or microdispensers configured to deliver two or more fragrances or scents from the same piece of jewelry.

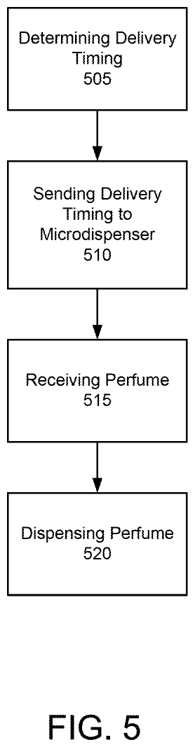

FIG. 5 illustrates a sample process for operating a piece of jewelry such as those described herein to deliver a fragrant liquid, such as perfume, to a person. A controller, such as controller 206 described above, may determine 505 delivery timing based upon a delivery schedule. For example, the controller may determine 505 timing information and information regarding an amount to apply from the delivery schedule. Based upon the delivery timing, the controller may send 510 instructions to a microdispenser to deliver one or more bursts of perfume. The microdispenser may receive 515 an amount of the perfume from a reservoir fluidly connected to the microdispenser. For example, the microdispenser may include an internal reservoir configured to hold a single dose, or a portion of a single dose, of the perfume. Once the microdispenser has received 515 the perfume, the microdispenser may dispense 520 the perfume onto the skin of the person wearing the jewelry.

As noted above, multiple pieces of jewelry (or a single piece of jewelry) may include multiple reservoirs and microdispensers. In such an arrangement, the process as described in FIG. 5 may be repeated for each reservoir and microdispenser pair. For example, a controller may determine 505 delivery timing for dispensing a second perfume based upon a second delivery schedule, the delivery timing including timing and dosage information for the second perfume. The controller may send 510 the instructions for the second perfume to a second microdispenser. The second microdispenser may receive 515 the second perfume from a second reservoir and dispense 520 the second perfume according to the delivery schedule. Depending upon the location of the second microdispenser, the second perfume may be applied at a different location than the first perfume.

As noted above, perfume microdispensers may be incorporated into various pieces of jewelry. To reduce the complication of integrating the specific functionality of the various components as described herein, multiple pieces of jewelry may be arranged or packaged as a set of jewelry, where various pieces of the jewelry have either smart or dumb functionality. For example, a set of jewelry may include a pair of earrings, a necklace, a bracelet, and a ring. Each piece of jewelry in the set may include a reservoir for holding an amount of perfume and at least one microdispenser fluidly connected to the reservoir. However, in certain implementations, a controller configured to control operation of each piece of jewelry may be included in only a single piece of jewelry. For example, the controller for the set of jewelry may be integrated into the necklace. The pieces of jewelry may then be arranged into a small area network using, for example, mesh network transceivers integrated into the controller (in the necklace) and the microdispensers (in the remaining pieces of jewelry). The pieces of jewelry may then be arranged in a master/slave arrangement where the controller in the necklace is configured to provide dispensing instructions to each of the other components. Such an arrangement may simplify the operation of the slave components. However, if the wearer opts to wear a portion of the jewelry set without the master piece (e.g., the necklace in this example), the microdispensers in the other pieces of jewelry may still have a manual dispensing control, allowing for dispensing of perfume from those components manually.

To continue the above example, the wearer may also have the ability to mix and match pieces of jewelry from different sets. Provided the pieces of jewelry are configured to communicate using a similar communication protocol, the wearer may be able to include earrings from a first jewelry set with a necklace from a second jewelry set. In such an arrangement, the necklace may still connect to and provide dispensing instruction to the earrings. This would provide a jewelry manufacturer with the ability to have an entire catalog of dispensing jewelry that is interoperable, thereby allowing consumers to mix and match jewelry while still benefiting from the perfume dispensing features.

Additionally, a psychological aspect may be incorporated into the techniques as described herein. Studies have shown that response to an aroma or odor may help to change a person's mood or attitude, and the field of aromatherapy has grown around this concept. The techniques as described herein may be used to provide aromatherapy relief to a person wearing the dispensing jewelry as well. For example, as noted above, the jewelry may include additional sensors. In certain implementations, a bracelet may include a heart rate monitor. If the heart rate monitor detects an increase in the wearer's pulse, the bracelet controller may cause the bracelet (or another piece of jewelry) to dispense a perfume or another fragrant liquid intended to calm or otherwise soothe the wearer in an attempt to reduce the wearer's pulse. Similarly, a piece of jewelry may also be configured to measure both heart rate and skin temperature. If the jewelry detects that a wearer's pulse and skin temperature have both dropped, the controller may determine that the wearer is getting drowsy and may dispense a perfume or other fragrant liquid intended to awaken or otherwise energize the wearer.

The devices and processes as described herein provide improved systems, devices, and processes for applying perfume using microdispensing jewelry. Such an approach overcomes the drawbacks of traditional perfume application systems, devices, and techniques, primarily over-dosage when initially applied and diminishing fragrance over time. Specific use cases of the devices and techniques as described above are briefly described in the following examples:

### Example 1

A person working in a large office building purchases a set of perfume dispensing earrings. The earrings are configured to connect to the person's smartphone using a Bluetooth connection. A smartphone application allows the person to control the amount of perfume to dispense and the frequency with which the perfume is dispensed. The person sets up a personalized schedule to dispense a small amount of perfume every half hour during the workday in order to maintain a fragrant aroma while not annoying or bothering coworkers as a result of over-dosing the perfume.

After work, the person intends to go on a date. The person can set the personalized schedule to increase the dosage amount and frequency during the date or can change the perfume being dispensed to another fragrance prior to the date, thereby altering their smell profile from the one that the person had during the workday.

### Example 2

A perfume company collaborates with a jewelry manufacturer to create a line of perfume dispensing jewelry. The perfume company designs a set of perfumes that have complimentary scents and packages the perfume in replaceable cartridges. The jewelry company designs and manufactures a set of jewelry. Each piece of jewelry is configured to receive the perfume cartridges and includes a microdispenser configured to dispense the perfume onto a person wearing the jewelry. The jewelry company also designs a piece of jewelry in the set to act as a master device, including a controller configured to provide dispensing instructions. The jewelry is packaged in various manners including a starter set (including the master device jewelry piece and various slave pieces of jewelry). The jewelry is also packaged such that a person can buy individual pieces of the jewelry.

The jewelry and perfume companies also collaborate with a technology company to make a smartphone application for use with the jewelry. The application provides an interface for the wearer of the jewelry to select the specific fragrance that is inserted into a piece of jewelry, the schedule at which to dispense the perfume, an order in which to dispense perfume (if using different fragrances), and other related parameters associated with the dispensing jewelry as described herein.

According to some examples, a personal ornament is described. The personal ornament may include a first container configured to store a first fragrant liquid; a first microdispenser fluidly connected to the container, where the first microdispenser is configured to deliver bursts of the first fragrant liquid over a period of time; and a controller operably connected to the first microdispenser and configured to provide instructions to the first microdispenser to deliver the first fragrant liquid according to a delivery schedule.

According to other examples, the first container may include a refillable reservoir. The refillable reservoir may also include a replaceable cartridge, and the replaceable cartridge may include the first fragrant liquid. The personal ornament may also include a manual control configured to cause the first microdispenser to deliver an additional burst of the first fragrant liquid. The controller may be further configured to modify the delivery schedule based upon an activity level of a person wearing the personal ornament. The controller may also be configured to modify the delivery schedule based upon at least one physiological signal of a person wearing the personal ornament. The personal ornament may further include at least one sensor configured to measure the at least one physiological signal of the person wearing the personal ornament. The at least one sensor may be configured to detect at least one of heart rate, skin temperature, and blood pressure.

According to further examples, the controller may include an input device configured to provide a person wearing the personal ornament with a capability to configure an amount of first fragrant liquid delivered by the first microdispenser. The first microdispenser may be configured such that the first microdispenser directs the burst of first fragrant liquid toward a portion of skin of a person wearing the personal ornament. The controller may be further configured to establish communication with a remote computing device. The controller may also be configured to receive an updated delivery schedule from the remote computing device. The remote computing device may be a smart phone, a wearable computer, a desktop computer, a laptop computer, a vehicle-mount computer, or a server. The first microdispenser may be configured to deliver the first fragrant liquid as a plurality of microdrops. Each of the plurality of microdrops may be about 25 picoliters to about 75 picoliters. The personal ornament may further include a second container configured to store a second fragrant liquid.

According to yet other examples, the personal ornament may further include a second microdispenser fluidly connected to the second container, where the second microdispenser is configured to deliver bursts of the second fragrant liquid. The controller may be operably connected to the second microdispenser and further configured to provide instructions to the second microdispenser to deliver the second fragrant liquid according to a second delivery schedule. The controller may also be operably connected to the second microdispenser and further configured to provide instructions to the second microdispenser to deliver the second fragrant liquid at substantially the same time as the first microdispenser delivers the first fragrant liquid, thereby layering the first fragrant liquid and the second fragrant liquid. The personal ornament may also include a power source. The power source may be a rechargeable battery. The rechargeable battery may be a kinetically chargeable battery. The rechargeable battery may also be an induction-charged battery. The first fragrant liquid may be a perfume. The first container may include a replaceable cartridge, and the replaceable cartridge may include the first fragrant liquid.

According to other examples, a method of dispensing a fragrant liquid on a person is described. The method may include determining, by a controller integrated into a personal ornament worn by the person, timing for dispensing a first fragrant liquid based upon a delivery schedule; sending, from the controller to a first microdispenser, instructions for the first microdispenser to deliver at least one burst of the first fragrant liquid; receiving, at the first microdispenser, the first fragrant liquid from a first container fluidly attached to the first microdispenser; and dispensing, by the first microdispenser, an amount of the first fragrant liquid onto a first portion of skin of the person.

According to some examples, the first container may include a refillable reservoir configured to store a quantity of the first fragrant liquid. The method may further include receiving, by the first microdispenser, an instruction from a manual control to deliver an additional burst of the first fragrant liquid. The method may also include modifying, by the controller, the delivery schedule based upon an activity level of the person; modifying, by the controller, the delivery schedule based upon at least one physiological signal of the person; establishing, by the controller, communication with a remote computing device; and/or receiving, by the controller, an updated delivery schedule from the remote computing device. The remote computing device may be a smart phone, a wearable computer, a desktop computer, a laptop computer, a vehicle-mount computer, or a server. The first microdispenser may be configured to dispense the first fragrant liquid as a plurality of microdrops. Each of the plurality of microdrops may be about 25 picoliters to about 75 picoliters.

According to other examples, the method may further include determining, by the controller, timing for dispensing a second fragrant liquid based upon a second delivery schedule; sending, from the controller to a second microdispenser, instructions for the second microdispenser to deliver at least one burst of the second fragrant liquid; receiving, at the second microdispenser, the second fragrant liquid from a second container fluidly attached to the second microdispenser; and dispensing, by the second microdispenser, an amount of the second fragrant liquid onto a second portion of skin of the person. The second microdispenser may deliver the second fragrant liquid at substantially the same time that the first microdispenser delivers the first fragrant liquid, thereby layering the first fragrant liquid and the second fragrant liquid. The first fragrant liquid may include a perfume. The first container may include a replaceable cartridge, and the replaceable cartridge may include the first fragrant liquid.

According to further examples, a system for dispensing a fragrant liquid on a person is described. The system may include a computing device comprising information related to a delivery schedule for dispensing a fragrant liquid on a person and a first personal ornament worn by the person. The first personal ornament may include a first container configured to store a first fragrant liquid, a first microdispenser fluidly connected to the first container, where the first microdispenser is configured to deliver bursts of the first fragrant liquid over a period of time, and a first controller operably connected to the first microdispenser. The first controller may be configured to establish communications with the computing device, receive the delivery schedule from the computing device, and provide instructions to the first microdispenser to deliver the first fragrant liquid according to the delivery schedule.

According to some examples, the computing device may be further configured to receive an updated delivery schedule from the person wearing the first personal ornament; and transmit the updated delivery schedule to the first controller. The first controller may be further configured to receive the updated delivery schedule from the computing device and provide updated instructions to the first microdispenser to deliver the first fragrant liquid according to the updated delivery schedule. The first container may include a refillable reservoir configured to store a quantity of the first fragrant liquid. The refillable reservoir may include a replaceable cartridge, where the replaceable cartridge may include the first fragrant liquid.

According to other examples, the first container may include a replaceable cartridge, and where the replaceable cartridge may include the first fragrant liquid. The first personal ornament may further include a manual control configured to cause the first microdispenser to deliver an additional burst of the first fragrant liquid. The first microdispenser may be configured to deliver the first fragrant liquid as a plurality of microdrops. Each of the plurality of microdrops may be about 25 picoliters to about 75 picoliters. The computing device may be a smart phone, a wearable computer, a desktop computer, a laptop computer, a vehicle-mount computer, or a server.

According to further examples, the system may further include a second personal ornament. The second personal ornament may include a second container configured to store a second fragrant liquid, a second microdispenser fluidly connected to the second container, where the second microdispenser is configured to deliver bursts of the second fragrant liquid over a period of time, and a second controller operably connected to the second microdispenser. The second controller may be configured to establish communications with the computing device, receive the delivery schedule from the computing device, and provide instructions to the second microdispenser to deliver the second fragrant liquid according to the delivery schedule. The delivery schedule may include timing information for delivery of the first fragrant liquid and the second fragrant liquid. Delivery of the first fragrant liquid may be at substantially the same time as delivery of the second fragrant liquid, thereby layering the first fragrant liquid and the second fragrant liquid. The first personal ornament may be selected from the group consisting of an earring, a necklace, a ring, a brooch, a hair clip, an armlet, a bracelet, a hair pin, a fascinator, a chatelaine, a cufflink, an anklet, a body chain, a pendant, a piercing, and a bangle. The second personal ornament may be selected from the group consisting of an earring, a necklace, a ring, a brooch, a hair clip, an armlet, a bracelet, a hair pin, a fascinator, a chatelaine, a cufflink, an anklet, a body chain, a pendant, a piercing, and a bangle.

According to yet other examples, a personal ornament is described. The personal ornament may include a first container configured to store a first fragrant liquid; a first microdispenser fluidly connected to the container, where the first microdispenser is configured to deliver bursts of the first fragrant liquid over a period of time; and a manual control operably connected to the first microdispenser and configured to cause the first microdispenser to deliver an additional burst of the first fragrant liquid.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the claims include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A personal ornament device comprising:
a first container configured to store a first fragrant liquid;
a first microdispenser fluidly coupled to the container, wherein the first microdispenser is configured to deliver bursts of the first fragrant liquid over a period of time; and
a controller operably coupled to the first microdispenser and configured to:
detect a physical activity level of a person wearing the personal ornament device; and
provide instructions to the first microdispenser to deliver the first fragrant liquid according to a delivery schedule based on the detected physical activity level of the person wearing the personal ornament device.

2. The personal ornament device of claim 1, wherein the first container comprises one or more refillable reservoirs.

3. (canceled)

4. The personal ornament device of claim 1, further comprising a manual control configured to cause the first microdispenser to deliver an additional burst of the first fragrant liquid.

5. (canceled)

6. The personal ornament device of claim 1, wherein the controller is further configured to modify the delivery schedule based upon one or more of a heart rate, a skin temperature, or a blood pressure of the person wearing the personal ornament device.

7. The personal ornament device of claim 6, further comprising at least one sensor configured to measure the one or more of the heart rate, the skin temperature, or the blood pressure of the person wearing the personal ornament.

8.-11. (canceled)

12. The personal ornament device of claim 1, wherein the controller is further configured to receive an updated delivery schedule from the remote computing device.

13. (canceled)

14. The personal ornament device of claim 1, wherein the first microdispenser is configured to deliver the first fragrant liquid as a plurality of microdrops, each of the plurality of microdrops having a volume from about 25 picoliters to about 75 picoliters.

15. (canceled)

16. The personal ornament device of claim 1, further comprising a second container configured to store a second fragrant liquid and a second microdispenser fluidly connected to the second container, wherein
the second microdispenser is configured to deliver bursts of the second fragrant liquid, and
the controller is further configured to provide instructions to the second microdispenser to deliver the second fragrant liquid according to a second delivery schedule.

17.-19. (canceled)

20. The personal ornament device of claim 1, further comprising a power source that includes one or more of a rechargeable battery, a kinetically chargeable battery, or an induction-charged battery.

21.-25. (canceled)

26. A method of dispensing a fragrant liquid on a person, the method comprising:
detecting a physical activity level of a person wearing a personal ornament device;
determining, by a controller integrated into the personal ornament device worn by the person, a delivery schedule based on the detected the physical activity level of the person wearing the personal ornament device;
determining, by the controller, timing for dispensing a first fragrant liquid based upon the delivery schedule;
sending, from the controller to a first microdispenser, instructions for the first microdispenser to deliver at least one burst of the first fragrant liquid;
receiving, at the first microdispenser, the first fragrant liquid from a first container fluidly attached to the first microdispenser; and
dispensing, by the first microdispenser, an amount of the first fragrant liquid onto a first portion of skin of the person.

27.-28. (canceled)

29. The method of claim 26, further comprising modifying, by the controller, the delivery schedule based upon the physical activity level of the person.

30. The method of claim 26, further comprising modifying, by the controller, the delivery schedule based upon one or more of a heart rate, a skin temperature, or a blood pressure of the person wearing the personal ornament device.

31. The method of claim 26, further comprising:
establishing, by the controller, communication with a remote computing device; and
receiving, by the controller, an updated delivery schedule from the remote computing device, wherein the remote computing device includes a smart phone, a wearable computer, a desktop computer, a laptop computer, a vehicle-mount computer, or a server.

32.-34. (canceled)

35. The method of claim 26, further comprising:
determining, by the controller, timing for dispensing a second fragrant liquid based upon a second delivery schedule;
sending, from the controller to a second microdispenser, instructions for the second microdispenser to deliver at least one burst of the second fragrant liquid;
receiving, at the second microdispenser, the second fragrant liquid from a second container fluidly attached to the second microdispenser; and
dispensing, by the second microdispenser, an amount of the second fragrant liquid onto a second portion of skin of the person at substantially the same time that the first microdispenser delivers the first fragrant liquid, thereby layering the first fragrant liquid and the second fragrant liquid.

36.-38. (canceled)

39. A system for dispensing a fragrant liquid on a person, the system comprising:
a computing device configured to store or generate a delivery schedule for dispensing a fragrant liquid on a person based on a detected physical activity level of the person; and
a first personal ornament device worn by the person, the first personal ornament device comprising:
a first container configured to store a first fragrant liquid,
a first microdispenser fluidly connected to the first container, wherein the first microdispenser is configured to deliver bursts of the first fragrant liquid over a period of time, and
a first controller operably connected to the first microdispenser and configured to:
establish communications with the computing device,
receive the delivery schedule from the computing device, and
provide instructions to the first microdispenser to deliver the first fragrant liquid according to the delivery schedule.

40. The system of claim 39, wherein
the computing device is further configured to:
receive an updated delivery schedule from the person wearing the first personal ornament device; and
transmit the updated delivery schedule to the first controller and
the first controller is further configured to:
receive the updated delivery schedule from the computing device, and
provide updated instructions to the first microdispenser to deliver the first fragrant liquid according to the updated delivery schedule.

41.-47. (canceled)

48. The system of claim 39, further comprising a second personal ornament device, the second personal ornament device comprising:
a second container configured to store a second fragrant liquid,
a second microdispenser fluidly connected to the second container, wherein the second microdispenser is configured to deliver bursts of the second fragrant liquid over a period of time, and
a second controller operably connected to the second microdispenser and configured to:
establish communications with the computing device,
receive the delivery schedule from the computing device, and
provide instructions to the second microdispenser to deliver the second fragrant liquid according to the delivery schedule.

49. The system of claim 48, wherein the delivery schedule comprises timing information for delivery of the first fragrant liquid and the second fragrant liquid.

50. The system of claim 49, wherein delivery of the first fragrant liquid is at substantially the same time as delivery of the second fragrant liquid, thereby layering the first fragrant liquid and the second fragrant liquid.

51. The system of claim 48, wherein the first personal ornament device and the second personal ornament device are selected from the group consisting of an earring, a necklace, a ring, a brooch, a hair clip, an armlet, a bracelet, a hair pin, a fascinator, a chatelaine, a cufflink, an anklet, a body chain, a pendant, a piercing, and a bangle.

52.-54. (canceled)
